(19) United States
(12) Patent Application Publication
(10) Pub. No.: US 2012/0196923 A1
(43) Pub. Date: Aug. 2, 2012

(54) POLYMERS FOR DELIVERING A SUBSTANCE INTO A CELL

(76) Inventors: Kaushal Rege, Chandler, AZ (US); Ravindra S. Kane, Niskayuna, NY (US); Steven M. Cramer, Schenectady, NY (US); Sutapa Barua, Tempe, AZ (US)

(21) Appl. No.: 13/318,384

(22) PCT Filed: May 17, 2010
(86) PCT No.: PCT/US10/35094

§ 371 Date: Jan. 13, 2012

Related U.S. Application Data
(60) Provisional application No. 61/178,654, filed on May 15, 2009.

(51) Int. Cl. A61K 31/7088; C07D 303/36; C07C 217/50; C12N 5/071

(52) U.S. Cl. 514/44 R; 435/375; 549/551; 564/504

(57) ABSTRACT

Disclosed herein are polymers that can be made cationic and used to deliver a substance into a cell. Also disclosed are pharmaceutical compositions comprising the polymers and methods of using the polymers.

### CROSS-REFERENCE TO RELATED APPLICATIONS

This application claims the benefit of priority to U.S. Provisional Application No. 61/178,654, filed on May 15, 2009, which is incorporated by reference herein in its entirety.

### STATEMENT REGARDING FEDERALLY SPONSORED RESEARCH OR DEVELOPMENT

This invention was made with U.S. Government support under grant no. CBET 0829128 awarded by the National Science Foundation. The government has certain rights in the invention.

### BACKGROUND

Delivery vehicles are often used to delivery a substance into a cell. Viruses, for example, are effective at delivering DNA into mammalian cells. However, concerns regarding safety, immunogenicity, repeated dosage, viral degradation, and production scale-up have motivated the investigation of nonviral approaches for delivering a substance into a cell. A variety of nonviral delivery vehicles have been discovered. For the delivery of anionic substances or substances that can be made anionic, such as therapeutic agents, peptides, polynucleic acids, and the like, cationic polymers are particularly useful. For example, cationic polymers can deliver exogenous DNA to cells and enhance the efficacy of virus-mediated gene transfer. A few examples of known cationic polymers used to deliver genes into cells include poly(L-lysine), poly(ethylene imine), chitosan, polyamidoamine or PAMAM dendrimers and poly(vinyl pyrrolidone).

Unfortunately, most cationic polymers exhibit high cytotoxicities. As a result, alternative delivery vehicles have been developed that exhibit lower cytotoxities, such as polymers based on cyclodextrin and carbohydrate comonomers, as well as genetically engineered protein-based polymers. To date, however, even the alternative delivery vehicles are not optimal and are not as effective as viral delivery vehicles in vivo. (Glover, D. J.; Lipps, H. J.; Jans, D. A. “Towards safe, non-viral therapeutic gene expression in humans.” Nat. Rev. Genet. 2005, 6 (4), 299-310.)

Thus, both the cytotoxicity and the low efficacy of nonviral delivery vehicles is a significant limitation in the development of safer alternatives to viral delivery vehicles. Accordingly, there exists a need for improved nonviral materials for delivering a substance into a cell that are both effective and safe. These needs and other needs are addressed by the present invention.

### SUMMARY

In accordance with the purposes of the disclosed materials, compounds, compositions, and methods, as embodied and broadly described herein, the disclosed subject matter, in one aspect, relates to polymers of diglycidylethers and amines that are useful for delivering a substance into a cell. In a further aspect, the disclosed subject matter relates to pharmaceutical compositions comprising both a polymer and a substance to be delivered into a cell. In still a further aspect, the disclosed subject matter relates to methods for delivering a substance into a cell using a polymer or pharmaceutical composition. In yet a further aspect, the disclosed subject matter relates to methods for treating a disorder by administering to a subject a polymer and a substance to be delivered into a cell, or by administering to a subject a pharmaceutical composition comprising a polymer and a substance to be delivered into a cell.

Additional advantages will be set forth in part in the description that follows, and in part will be obvious from the description, or can be learned by practice of the aspects described below. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1a is a reaction scheme employed for the generation of the library of eighty cationic polymers. FIG. 1b is a list of the diglycidyl ethers used in the combinatorial matrix of the polymer library. FIG. 1c is a list of the amines used in the combinatorial matrix of the polymer library.

FIG. 2 is a three-dimensional bar plot showing DNA-binding activity of the diglycidyl ether based cationic polymer library determined using the ethidium bromide displacement assay. The percent fluorescence decreased upon polymer binding to calf Thymus DNA intercalated with Ethidium Bromide was used to measure polymer efficacy.

FIGS. 3a-c are plots demonstrating transfection of mammalian cells using polymer leads selected from the DNA binding screen (Example 2). Polymer transfection efficacies are reported as relative to that of pEI-25. Relative efficacy data are plotted on a logarithmic scale (y-axis) and statistical significance using p-values was determined by comparing data for a given polymer with pEI-25. (3a) Polymer-mediated transfection of PC3-PSMA cells in the absence of serum using a polymer: pGL3 plasmid ratio of 25:1 (w/w). (3b) Transfection of PC3-PSMA cells in the absence of serum using different polymer: plasmid ratios of the 1,4C-1,4 Bis polymer. (3c) Polymer-mediated transfection of PC3-PSMA cells in the presence of 10% fetal bovine serum using a polymer: pGL3 plasmid ratio of 25:1. (3d) Polymer-mediated transfection of murine osteoblasts using 1,4C-1,4 Bis and pEI-25 polymers in the presence of 10% fetal bovine serum using a polymer:pGL3 plasmid ratio of 25:1.

FIG. 4 is a plot displaying the percentage of dead PC3-PSMA cells following polymer/polyplex treatment (Example 4).

FIG. 5a is a plot of polymerization kinetics of EGDE-3,3′ and 1,4C-1,4Bis polymer formation as reported by the disappearance of amines as a function of time. FIG. 5b is a plot obtained from fourier transform infrared (FTIR) spectroscopy of 1,4C-1,4 Bis at polymerization at time 0 h (monomer mixture) and 16 h following initiation of the polymer reaction.

### DETAILED DESCRIPTION

The materials, compounds, compositions, articles, devices, and methods described herein can be understood more readily by reference to the following detailed description of specific aspects of the disclosed subject matter and the Examples included therein and to the Figures.

Before the present materials, compounds, compositions, and methods are disclosed and described, it is to be understood that the aspects described below are not limited to specific synthetic methods or specific reagents, as such can, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a cationic polymer” includes mixtures of two or more such cationic polymers, reference to “a substance” includes mixtures of two or more such substances, reference to “a composition” includes mixtures of two or more such compositions, and the like.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

“Optional” or “optionally” means that the subsequently described event or circumstance can or can not occur, and that the description includes instances where said event or circumstance occurs and instances where it does not.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OA¹ where A¹ is alkyl as defined above.

The term alkoxylalkyl as used herein is an alkyl group that contains an alkoxy substituent and can be defined as -A¹-O-A², where A¹ and A² are alkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiA¹A²A³, where A¹, A², and A³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfo-oxo” as used herein is represented by the formulas —S(O)A¹, —S(O)₂A¹, —OS(O)₂A¹, or —OS(O)₂OA¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above. Throughout this specification “S(O)” is a short hand notation for S═O.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “sulfone” as used herein is represented by the formula A¹S(O)₂A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “sulfoxide” as used herein is represented by the formula A¹S(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The terms “nucleic acid,” “polynucleic acid,” and “polynucleotide,” as used herein, refer to a polymer comprising at least two residues of a nucleotide, which can include any N-glycoside or C-glycoside of a purine or pyrimidine base or of a modified purine or pyrimidine base, which includes those bases that do not occur naturally. Specific examples of such polymers include without limitation any form of ribonucleic acid (RNA), deoxyribonucleic acid (DNA), genomic DNA, messenger RNA (mRNA), complementary DNA (cDNA), antisense RNA (aRNA), a synthetic nucleic acid polymer, or a mixture thereof, among others.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Reference will now be made in detail to specific aspects of the disclosed materials, compounds, compositions, articles, and methods, examples of which are illustrated in the accompanying Examples and Figures.

As briefly discussed above, the disclosed subject matter, in one aspect, relates to polymers that are useful for delivering a substance into a cell. The disclosed polymers are both effective at delivering substances into a cell and safe (i.e., not undesirably cytotoxic).

Polymers

Generally, the disclosed polymers are polymers of diepoxide and an amine, such as a diamine. The polymers are generally branched at one or more points on the polymer backbone. The amines of the polymers allow the polymers to be made cationic by subjecting the polymers to acid. Thus, by reference to “polymers” herein, the cationic forms are also contemplated. The cationic forms of the polymers can then bind to a variety of substances that can be delivered into a cell.

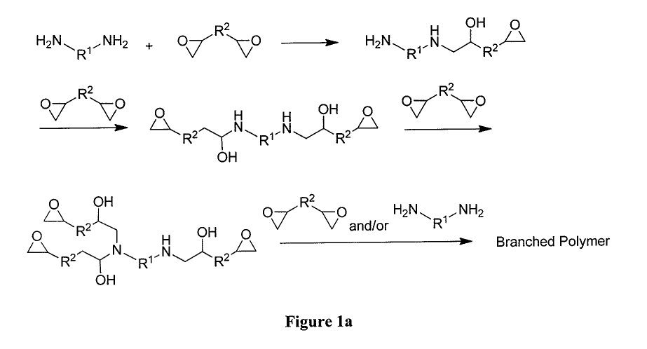

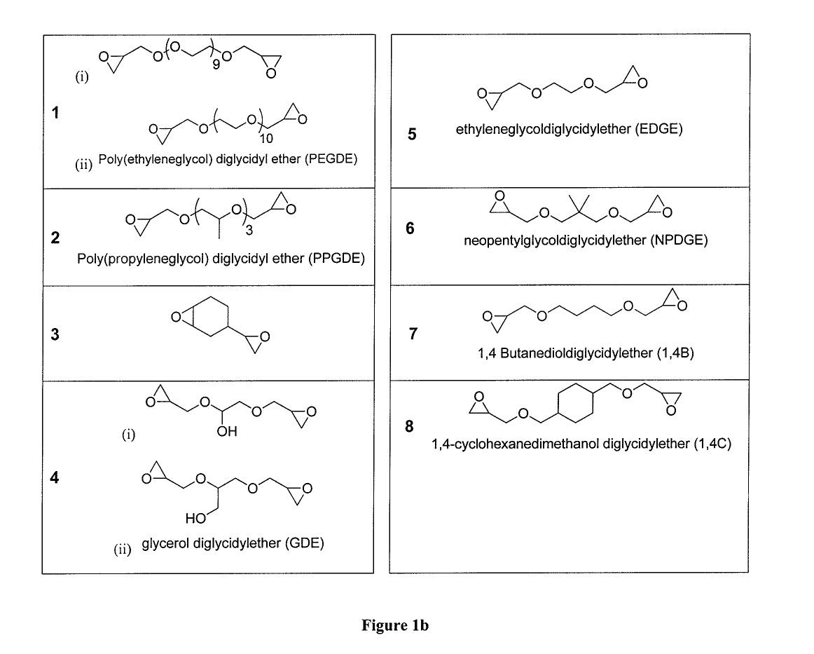

With reference to FIG. 1a, in one aspect, the polymers disclosed herein are polymers of amines and diepoxides, wherein the amine is represented by the formula:

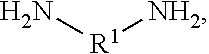

and wherein the diepoxide is represented by a formula:

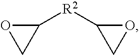

wherein R¹ and R² is independently optionally substituted alkyl, optionally substituted cycloalkyl, optionally substituted heteroalkyl, optionally substituted heterocycloalkyl, optionally substituted aryl, optionally substituted heteroaryl, or a combination thereof.

In specific aspects, R¹ is optionally substituted alkyl or heteroalkyl, such as aminoalkyl. Specific examples of amines, showing suitable variations in R¹, are shown in FIG. 1c. In other specific aspects, R² is optionally substituted alkyl, optionally substituted heteroalkyl, such as ethylene glycol or polyethylene glycol, optionally substituted cycloalkyl, or optionally substituted heterocycloalkyl. Specific examples of epoxides, showing suitable variations in R², are shown in FIG. 1b.

The polymers are prepared from monomers having two or more reactive functionalities, namely epoxides and amines, and thus ultimately have a variety of structures that are generally branched, have one or more amines, including secondary and tertiary amines in the polymer backbone and primary amines as end groups, one or more secondary alcohols (from the ring-opening of the epoxide), and/or one or more epoxides as endgroups.

With reference to FIG. 1a, the polymers are prepared by reacting the amine with the diepoxide, which induces a polymerization sequence, such as the exemplary sequence shown in FIG. 1a, wherein either an amine end group in a growing chain reacts with another monomeric diglycidyl either, or a monomeric amine reacts with a glycidyl ether endgroup, or both. The ratio of amines, glycidyl ethers, and alcohols in the backbone can be modulated by the stoichiometric ratio of the monomers. The molecular weight and structure of the polymer can be likewise modulated by not only the monomer ratio but also by polymerization conditions, such as temperature or duration.

In practice, the polymers can be made by reacting the monomers in solution or neat. When it is desired to form cationic polymers from the polymers, acid can be added to the polymers to protonate one or more amines of the polymer. If the polymer is in solution, only a slight pH modification is needed. Lowering the pH to about 7.4, for example, from a more basic starting point, using acid will suffice to protonate a sufficient number of amines.

With reference to FIG. 1b and FIG. 1c, specific examples of the disclosed polymers include polymers produced from any combination of monomers 1 through 8 and monomers A-J, including copolymers 1(i) A, 1(i) B, 1(i) C, 1(i) D, 1(i) E, 1(i) F, 1(i) G, 1(i) H, 1(i) I, 1(i) J; 1(ii) A, 1(ii) B, 1(ii) C, 1(ii) D, 1(ii) E, 1(ii) F, 1(ii) G, 1(ii) H, 1(ii) I, 1(ii) J; 2A, 2B, 2C, 2D, 2E, 2F, 2G, 2H, 2I, 2J; 3A, 3B, 3C, 3D, 3E, 3F, 3G, 3H, 3I, 3J; 4(i)A, 4(i)B, 4(i)C, 4(i)D, 4(i)E, 4(i)F, 4(i)G, 4(i)H, 4(i)I, 4(i)J; 4(ii)A, 4(ii)B, 4(ii)C, 4(ii)D, 4(ii)E, 4(ii)F, 4(ii)G, 4(ii)H, 4(ii)I, 4(ii)J; 5A, 5B, 5C, 5D, 5E, 5F, 5G, 5H, 5I, 5J; 6A, 6B, 6C, 6D, 6E, 6F, 6G, 6H, 6I, 6J; 7A, 7B, 7C, 7D, 7E, 7F, 7G, 7H, 7I, 7J; 8A, 8B, 8C, 8D, 8E, 8F, 8G, 8H, 8I, 8J, and combinations thereof. In a specific aspect, the polymer is poly(ethyleneglycol diglycidyl ether)-co-(3,3′-diamino-N-methyl dipropylamine) or poly(1,4-cyclohexanedimethanol diglycidyl ether)-co-[1,4-bis(3-aminopropyl)piperazine]. In one aspect, the polymer is not 1(i) A, 1(i) B, 1(i) C, 1(i) D, 1(i) E, 1(i) F, 1(i) G, 1(i) H, 1(i) I, 1(i) J. In another aspect, the polymer is not 4(i)A, 4(i)B, 4(i)C, 4(i)D, 4(i)E, 4(i)F, 4(i)G, 4(i)H, 4(i)I, 4(i)J.

The disclosed polymers can have a number average molecular weight (Mn) of from about 1 to about 20 kDa, including for example, 1, 2, 3, 4, 10, 15, or 18. The disclosed polymers can have a weight average molecular weight (Mw) of from about 10 to about 50 kDa, including for example, 15, 20, 23, 26, 30, 35, 40, or 45. The polydispersity (Mw/Mn) of the disclosed polymers can be from about 2 to about 7, including for example, about 3, 4 or 6.

The polymers can be tested to evaluate their usefulness as delivery agents for substances to be delivered into a cell using screening techniques known in the art. For example, to evaluate the usefulness of the polymers in delivering an anionic polynucleic acid, such as DNA, into a cell, DNA-binding efficacies of the polymers can be determined using the ethidium bromide displacement assay, discussed below. In vitro and/or in vivo evalutation of the polymers can be likewise evaluated by any method known in the art.

Contemplated uses of the polymers include delivering a variety of substances into a cell. As noted above, the disclosed polymers and their cationic forms can form an association with a substance that has an affinity therefore, and as such can function as a delivery vehicle for delivering the substance into a cell. A variety of drugs, bioactive agents, biomolecules, such as peptides, proteins, nucleic acids, polynucleic acids, polynucleotides, among others, which associate or can be caused to associate with a disclosed cationic polymer, can be delivered into a cell. Peptides and proteins include any polymer of at least two residues of a natural or non-natural amino acid.

In one aspect, the disclosed polymers can be used in transfection procedures. Accordingly, the disclosed polymers can be used to facilitate the intercellular delivery of DNA or RNA sequences, for example, sequences coding for therapeutically active polypeptides. The disclosed polymers can also be used to deliver a primer, promoter, TAG, or siRNA to a cell. Likewise, disclosed polymers can be similarly used for the delivery of an expressed gene product, such as the polypeptide or protein itself. Thus, polymer-mediated delivery of DNA and RNA polynucleotides or proteins can provide therapy for genetic diseases by interfering with known sequences or cellular activities, or by supplying deficient or absent gene products to treat any genetic disease in which the defective gene or its product has been identified. The polymer-mediated intracellular delivery described above can also provide immunizing polypeptides to the cell, for example, by delivering a polynucleotide coding for the immunogen, or by delivering the immunogen itself.

Other therapeutically important nucleic acids suitable for polymer-mediated delivery are negatively charged oligonucleotides including antisense polynucleotide sequences, useful in eliminating or reducing the production of a gene product, as described by Tso, P. et al. Annals New York Acad. Sci. 570:220-241 (1987). Also disclosed is the delivery, by means of the polymer, of ribozymes, or catalytic RNA species, for example, the “hairpin” type as described by Hampel et al. Nucleic Acids Research 18(2):299-304 (1990); or the “Hammerhead” type described by Cech. T. and Bass, B. Annual Rev. Biochem. 55:599-629 (1986). These antisense nucleic acids or ribozymes can be expressed (replicated) in the transfected cells. The DNA sequences delivered can be those sequences that do not integrate into the genome of the host cell or those that do integrate into the genome of the host. These can be non-replicating DNA sequences, or specific replicating sequences genetically engineered to lack the genome-integration ability.

When the nucleic acid to be delivered is mRNA, it can be readily prepared from the corresponding DNA in vitro. For example, conventional techniques utilize phage RNA polymerases SP6, T3, or T7 to prepare mRNA from DNA templates in the presence of the individual ribonucleoside triphosphates. An appropriate phage promoter, such as T7 origin of replication site is placed in the template DNA immediately upstream of the gene to be transcribed. Systems utilizing T7 in this manner are well known, and are described in the literature, e.g., in Current Protocols in Molecular Biology, §3.8 (vol. 1, 1988).

In addition, disclosed herein is the delivery of mRNA that is chemically blocked at the 5′ and/or 3′ end to prevent access by RNase (this enzyme is an exonuclease and therefore does not cleave RNA in the middle of the chain). Such chemical blockage can substantially lengthen the half life of the RNA in vivo. By adding a group with sufficient bulk to the RNA, access to the chemically modified RNA by RNASE can be prevented.

Other therapeutic uses of the polymers include the delivery of nucleoside or nucleotide analogues having an antiviral effect, such as dideoxynucleotides, didehydronucleotides, nucleoside or nucleotide analogues having halo-substituted purine or pyrimidine rings such as 5-trifluoromethyl-2-deoxyuridine or 5-flurouracil; nucleoside or nucleotide analogues having halo- and azido-substituted ribose moieties, such as 3′-azido-3′deoxythymidine (AZT), nucleoside analogues having carbon substituted for oxygen in the ribose moiety (carbocyclic nucleosides), or nucleotide analogues having an acyclic pentose such as acyclovir or gancyclovir (DHPG). The antiviral potency of these analogues is found to be increased when they are presented to the cells as phospholipid derivatives. Effective antiviral lipid derivatives of nucleoside analogues comprise phosphatidyl 2′,3′-dideoxynucleosides, 2′,3′-didehydronucleosides, 3-azido-2-deoxynucleosides, 3-fluorodeosynucleosides and 3′-fluorodideoxynucleosides, 9-β-D-arabinofuranosyladenine (araA), 1-β-D-arabinofuranosylcytidine (araC), nucleosides such as acyclovir and gancyclovir having an acyclic ribose group, or the same nucleoside analogues as diphosphate diglyceride derivatives.

Among other therapeutically important agents that can be delivered are peptides comprising physiologic species such as interleukin-2, tumor necrosis factor, tissue plasminogen activator, factor VIII, erythropoietin, growth factors such as epidermal growth factor, growth hormone releasing factor, neural growth factor, and hormones such as tissue insulin, calcitonin, and human growth hormone as well as toxic peptides such as ricin, diphtheria toxin, or cobra venom factor, capable of eliminating diseased or malignant cells.

Use of the disclosed polymers is also contemplated for the intra-cellular delivery of various other agents according to methods known to those skilled in the art, for example as described in Duzgunes, N., Subcellular Biochemistry 11:195-286 (1985). Materials to be delivered can be proteins or polypeptides, as discussed above, or other negatively charged molecules, monoclonal antibodies, RNA-stabilizing factors and other transcription and translation regulating factors, antisense oligonucleotides, ribozymes, and any molecule possessing intracellular activity that can also associate with or be caused to associate with a disclosed cationic polymer. Polymer-mediated delivery further protects the described agents from non-productive sequestration by substances of the extracellular environment.

The delivery procedures described herein can be carried out in vitro, in vivo, or ex vivo. Thus, a polymer and a substance to be delivered into a cell can be administered directly into a subject, as will be discussed below. Alternatively, a cell can be treated with a polymer and a substance to be delivered into the cell, followed by introducing the treated cell into a subject to thereby treat a disorder. In another alternative, a cell of a living organism can be removed from the organism, treated with a polymer and a substance to be delivered into the cell, followed by reintroduction of the treated cell into the organism to thereby treat a disorder.

In one aspect, the polymer and/or the substance to be delivered into the cell can be present in a pharmaceutical composition. Local or systemic delivery of the substance can be achieved by administration comprising application or insertion of the pharmaceutical composition into body cavities, inhalation or insufflation of an aerosol, or by parenteral introduction, comprising intramuscular, intravenous, intradermal, peritoneal, subcutaneous and topical administration. The nucleic acids, for example, can be delivered to the interstitial space of tissues of the animal body, including those of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. The effect of the polymers in these pharmaceutical compositions is to enhance the potency and efficiency of the therapeutic agent contained therein by facilitating its intracellular delivery.

In all of the nucleic acid delivery strategies disclosed herein, an effective DNA or mRNA dosage will generally be in the range of from about 0.02 μg/kg to about 100 mg/kg, usually about 0.005-5 mg/kg. However, as will be appreciated, this dosage will vary in a manner apparent to those of skill in the art according to, e.g., the activity of the peptide coded for by the nucleic acid.

Topical formulations are those advantageously applied to the skin or mucosa. Target mucosa can be that of the gastrointestinal tract, comprising the mouth, naso-pharynx and stomach. Other target tissues can be the accessible surfaces and canal of the ear and the ocular tissues. Polymers present in topical formulations can act to facilitate introduction of bioactive molecules into the target tissue, such as the stratum corneum of the skin, by perturbing the barrier properties of the protective membrane, or by introducing perturbing agents or penetration enhancers such as Azone™ or by promoting the activity of these penetration enhancers. They can also be delivered into muscle or skin using a vaccine gun.

Other topical compositions comprising the polymers are preparations comprising topical antibiotics such as clindamycin, tobramycin, neomycin, gentamycin, tetracycline, erythromycin; oxidants such as benzoyl peroxide, antifungal agents, such as clotrimazole, miconazole, nystatin, lactoconzole, econazole, and tolnaftate; retinoic acid for the treatment of herpes simplex and comprising antiviral nucleoside analogues such as acyclovir and gancyclovir.

Other formulations comprising the disclosed polymers are topical preparations containing an anesthetic or cytostatic agent, immunomodulators, bioactive peptides or oligonucleotides, sunscreens or cosmetics. Preparations for topical use are conveniently prepared with hydrophilic and hydrophobic bases in the form of creams, lotions, ointments or gels; alternatively, the preparation can be in the form of a liquid that is sprayed on the skin. The effect of the cationic polymers is to facilitate the penetration of the active antiviral agent through the stratum corneum of the dermis.

Similar preparations for ophthalmic use are those in which the pharmacologically effective agent is timolol, betaxolol, levobunaloa, pilocarpine, and the antibiotics and corticosteriods disclosed for topical applications.

The composition and form of pharmaceutical preparations comprising the polymers disclosed, in combination with a drug or other therapeutic agent, can vary according to the intended route of administration.

Orally administered preparations can be in the form of solids, liquids, emulsions, suspensions, or gels, or preferably in dosage unit form, for example as tablets or capsules. Tablets can be compounded in combination with other ingredients customarily used, such as tale, vegetable oils, polyols, gums, gelatin, starch, and other carriers. The cationic polymers can be dispersed in or combined with a suitable liquid carrier in solutions, suspensions, or emulsions.

Parenteral compositions intended for injection, either subcutaneously, intramuscularly, or intravenously, can be prepared as liquids or solid forms for solution in liquid prior to injection, or as emulsions. Such preparations are sterile, and liquids to be injected intravenously should be isotonic. Suitable excipients are, for example, water, dextrose, saline, and glycerol.

Administration of pharmaceutically acceptable salts of the substances described herein is included within the scope of the invention. Such salts can be prepared from pharmaceutically acceptable non-toxic bases including organic bases and inorganic bases. Salts derived from inorganic bases include sodium, potassium, lithium, ammonium, calcium, magnesium, and the like. Salts derived from pharmaceutically acceptable organic non-toxic bases include salts of primary, secondary, and tertiary amines, basic amino acids, and the like. For a helpful discussion of pharmaceutical salts, see S. M. Berge et al., Journal of Pharmaceutical Sciences 66:1-19 (1977) the disclosure of which is hereby incorporated by reference.

Substances for injection, a preferred route of delivery, can be prepared in unit dosage form in ampules, or in multidose containers. The substances to be delivered can be present in such forms as suspensions, solutions, or emulsions in oily or preferably aqueous vehicles. Alternatively, the salt of the substance can be in lyophilized form for reconstitution, at the time of delivery, with a suitable vehicle, such as sterile pyrogen-free water. Both liquids as well as lyophilized forms that are to be reconstituted will comprise agents, preferably buffers, in amounts necessary to suitably adjust the pH of the injected solution. For any parenteral use, particularly if the formulation is to be administered intravenously, the total concentration of solutes should be controlled to make the preparation isotonic, hypotonic, or weakly hypertonic. Nonionic materials, such as sugars, are preferred for adjusting tonicity, and sucrose is particularly preferred. Any of these forms can further comprise suitable formulatory agents, such as starch or sugar, glycerol or saline. The compositions per unit dosage, whether liquid or solid, can contain from 0.1% to 99% of polynucleotide material.

Also disclosed are kits comprising the polymers and the substance to be delivered into the cell. The kits can comprise one or more packaged unit doses of a composition comprising the polymer and the substance to be delivered into the cell. The units dosage ampules or multidose containers, in which the polymer and the substance to be delivered are packaged prior to use, can comprise an hermetically sealed container enclosing an amount of polynucleotide or solution containing a substance suitable for a pharmaceutically effective dose thereof, or multiples of an effective dose. The polymer and substance can be packaged as a sterile formulation, and the hermetically sealed container is designed to preserve sterility of the formulation until use.

The disclosed polymers can also be present in liquids, emulsions, or suspensions for delivery of active therapeutic agents in aerosol form to cavities of the body such as the nose, throat, or bronchial passages. The ratio of active ingredient to the polymer and the other compounding agents in these preparations will vary as the dosage form requires.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, gels, or the like, preferably in unit dosage form suitable for single administration of a precise dosage. The compositions will include, as noted above, an effective amount of the selected lipocomplex in combination with a pharmaceutically acceptable carrier and, in addition, can include other medicinal agents, pharmaceutical agents, carriers, adjuvants, diluents, etc.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, etc., an active compound as described herein and optional pharmaceutical adjuvants in an excipient, such as, for example, water, saline aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art; for example see Remington's Pharmaceutical Sciences, referenced above.

Parental administration, if used, is generally characterized by injection. Injectables can be prepared in conventional forms, either as liquid solutions or suspensions, solid forms suitable for solution or suspension in liquid prior to injection, or as emulsions. A more recently revised approach for parental administration involves use of a slow release or sustained release system, such that a constant level of dosage is maintained. See, e.g., U.S. Pat. No. 3,710,795, which is incorporated by reference herein.

When the polymers and substances to be delivered into a cell are used in subjects, the subject can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. Data are reported as mean±one standard deviation of independent replicate experiments. Statistical significance was determined for a given polymer using the unpaired Student's t-test.

### Example 1

### Synthesis of Cationic Polymers

Eight diepoxides, 1,4 butanediol diglycidyl ether (1,4 B), 1,4-cyclohexanedimethanol diglycidyl ether (1,4 C), 4-vinylcyclohexene diepoxide (4VCD), ethyleneglycol diglycidyl ether (EDGE), glycerol diglycidyl ether (GDE), neopentylglycol diglycidyl ether (NPDGE), poly(ethyleneglycol) diglycidyl ether (PEGDE), and poly(propyleneglycol) diglycidyl ether (PPGDE) were purchased from Sigma-Aldrich and were used without any further purification. Ten amines, 1-(2-aminoethyl)piperidine, 1,4-bis(3-aminopropyl)piperazine, (1,4 Bis), 3,3′-diamino-N-methyl dipropylamine(3,3′), 4,7,10-trioxa-1,13-tridecanediamine, aniline, butylamine, diethylenetriamine (DT), ethylenediamine (ED), N-(2-amino ethyl)-1,3-propanediamine (N-2amino), and pentaethylenehexamine were also purchased from Sigma-Aldrich and used as received.

FIG. 1a shows the general reaction scheme employed for the generation of the library of eighty cationic polymers. FIG. 1b and FIG. 1c show monomers used in developing the combinatorial matrix of the polymer library. The eight diglycidyl ethers (2.3 mmol) were reacted with equimolar amounts of the amines; neat as-purchased solutions were employed for both reactants. In the case of pentaethylenehexamine, the low solubility of the resulting polymers at a 1:1 ratio of diglycidyl ether to amine necessitated the use of a 10:1 diglycidyl ether:amine molar ratio in subsequent experiments. The polymerization was carried out in 20 mL glass scintillation vials for 16 h. After 16 h, the resulting polymer was diluted to a concentration of 2 mg/mL in 20 mM Tris buffer, pH 7.4. The solution pH was adjusted to 7.4 using 30% hydrochloric acid in de-ionized (DI) water to compensate for the alkalinity of the polymers. Polymers that were soluble at a concentration of 2 mg/mL at pH 7.4 were evaluated for their DNA-binding efficacies. Sixteen out of the eighty polymers synthesized were not soluble at concentrations of 2 mg/ml. Sixty-four soluble polymers were employed in the primary screening which involved an evaluation of their respective DNA-binding efficacies using the ethidium bromide displacement assay.

### Example 2

### Screening of DNA-Binding Activity

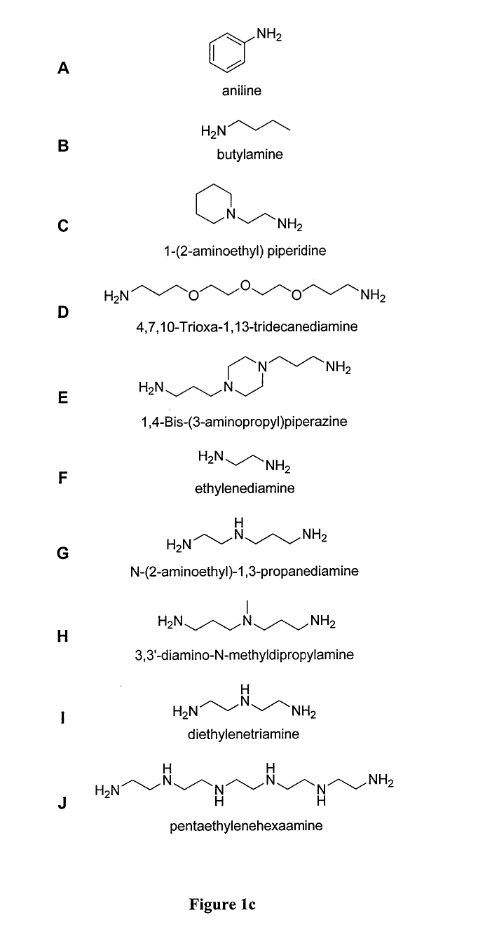

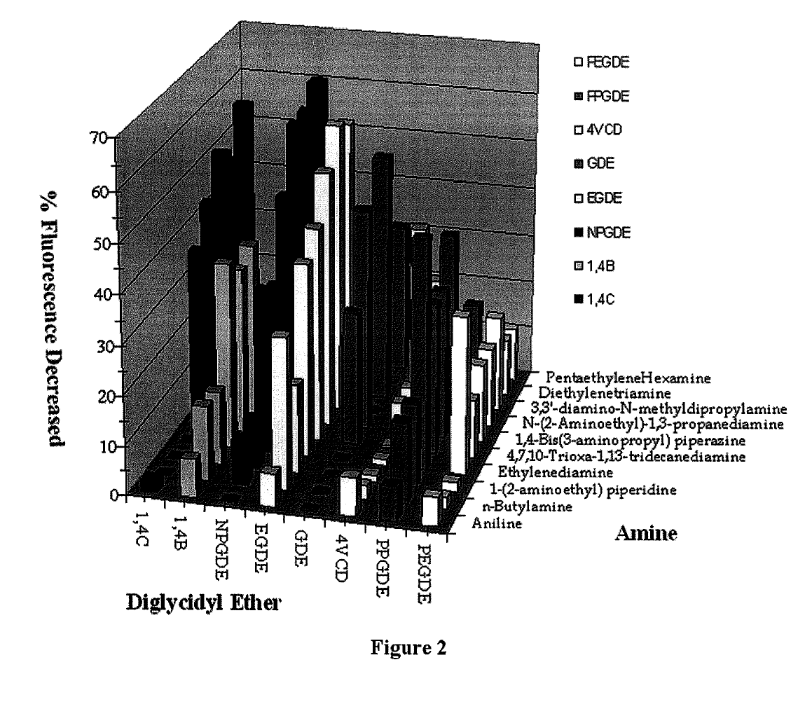

An ethidium bromide displacement assay was employed to evaluate the DNA-binding affinity of the cationic polymer library in parallel. 1.5 mL of 6 μg/mL double-stranded, calf-thymus DNA was equilibrated with 15 μL of 0.5 mg/mL ethidium bromide (all solutions were prepared in 20 mM Tris buffer, pH 8.0). After equilibration, 25 μL of 2 mg/mL polymer was added to the DNA-ethidium bromide mixture and equilibrated for 20 minutes. 150 μL of the polymer-DNA solution was transferred into a 96-well microtiter plate and the fluorescence (excitation at 260 nm, emission at 595 nm) was measured using a plate reader (Perkin Elmer Lambda 6.0). The decrease in fluorescence intensity (percent fluorescence decreased compared to control) was used to rank DNA-binding efficacies of individual polymers. FIG. 2 shows the DNA-binding efficacy of the cationic polymer library determined using the ethidium bromide displacement assay. As expected, polymers based on monoamines (e.g., aniline and butylamine) demonstrated low values of percent fluorescent decreased (i.e., low DNA-binding efficacies) while those derived from higher homologue polyamines, such as 1,4-Bis(3-aminopropyl)piperazine, 3,3′-diamino-N-methyl dipropylamine, diethylenetriamine, and N-(2-aminoethyl)-1,3-propanediamine demonstrated higher efficacies. Seven representative polymer leads, with different DNA binding efficacies (percent fluorescence decreased values ranging from 30% to 60%), were chosen for the transfection of PC3-PSMA cells as described below. The following references describe the ethinium bromide displacement assay in detail: Boger, D. L.; Fink, B. E.; Brunette, S. R.; Tse, W. C.; Hedrick, M. P. A simple, high-resolution method for establishing DNA binding affinity and sequence selectivity. Journal of the American Chemical Society 2001, 123 (25), 5878-91; Geall, A. J.; Blagbrough, I. S. Rapid and sensitive ethidium bromide fluorescence quenching assay of polyamine conjugate-DNA interactions for the analysis of lipoplex formation in gene therapy. Journal of Pharmaceutical and Biomedical Analysis 2000, 22 (5), 849-59; Rege, K.; Hu, S.; Moore, J. A.; Dordick, J. S.; Cramer, S. M. Chemoenzymatic synthesis and high-throughput screening of an aminoglycoside-polyamine library: identification of high-affinity displacers and DNA-binding ligands. Journal of the American Chemical Society 2004, 126 (39), 12306-15; Rege, K.; Ladiwala, A.; Hu, S.; Breneman, C. M.; Dordick, J. S.; Cramer, S. M. Investigation of DNA-binding properties of an aminoglycoside-polyamine library using quantitative structure activity relationship (QSAR) models Journal of Chemical Information and Modeling 2005, 45 (6), 1854-63; each of which is incorporated herein by this reference for teachings of the ethinium bromide displacement assay.

### Example 3

### Cationic Polymer-Mediated Transfection

The pGL3 control vector (Promega Corp., Madison, Wis., U.S.A.), which encodes for the modified firefly luciferase protein under the control of an SV40 promoter, was used in transfection experiments. The PC3-PSMA human prostate cancer cell line was provided by Dr. Michel Sadelain of the Memorial Sloan Cancer Center, New York, N.Y., U.S.A. The cells were cultured in a 5% CO₂ incubator at 37° C. using RPMI-1640 medium containing 10% heat-inactivated fetal bovine serum (FBS) and 1% antibiotics (10,000 units/mL penicillin G/10,000 μg/mL streptomycin). MC3T3 murine osteoblasts were cultured in a 5% CO₂ incubator in Dulbecco's Modified Eagle's Medium (DMEM; BioWhittaker®) containing 4.5 g/L glucose and L-glutamine, supplemented with 10% fetal bovine serum (Invitrogen, Calif., U.S.A.) and 1% penicillin/streptomycin (Invitrogen, Calif., U.S.A.). PC3-PSMA and MC3T3 cells were seeded in 24-well plates at a density of 50,000 cells/well and allowed to attach overnight. Polymer:pGL3 control plasmid at weight ratios of 25:1 (polymer concentration 10 ng/μL and 200 ng pGL3 plasmid in each well) were incubated for 30 minutes at room temperature and the resulting polyplexes were added to cells for 6 h either in the absence or presence of serum (10% FBS), at the end of which, fresh serum-containing medium was added to the cells. Following further incubation for 48 h, cells were lysed using the Bright Glo kit (Promega) and analyzed for luciferase protein expression (in relative luminescence units or RLU) using a plate reader (Bio-Tek Synergy 2). The protein content in each well was determined using the BCA assay and the luminescence value (RLU) was normalized by the protein content. Transfection efficacies of different polymers from the library were compared with the normalized value (RLU/mg protein) obtained for pEI-25.

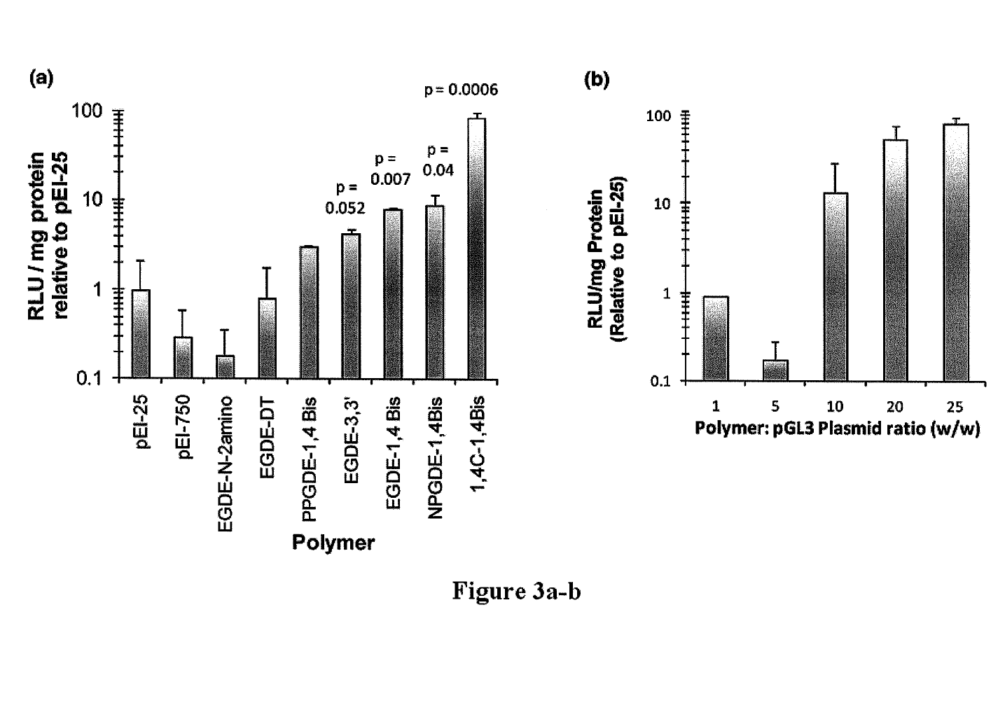

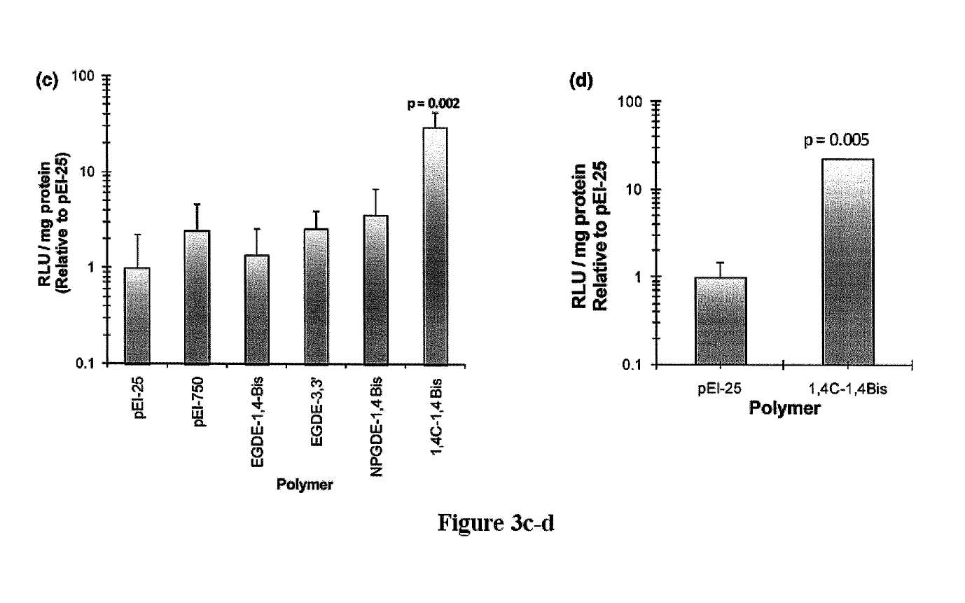

FIG. 3a shows the transfection of PC3-PSMA cells with the pGL3 plasmid using a set of lead polymers selected from the DNA-binding screen. Representative polymer leads that possessed moderate (30% fluorescence decreased) to high (>60% fluorescence decreased) DNA binding efficacies were employed in the transfection experiments. Calf-thymus DNA was used only as generic double-stranded DNA in the primary DNA binding screen for identifying lead polymers. However, the lack of a constitutive promoter region in calf-thymus DNA implies that this DNA cannot be employed as a reporter for transfection. Consequently, transfections were carried out with the pGL3 control vector which codes for luciferase protein. A polymer to plasmid ratio of 25:1 was employed in order to evaluate the transfection efficacies of the selected polymers. The use of nitrogen: phosphorus (N:P) ratio is common in comparing cationic lipid and cationic polymer meditated gene delivery. However, a w/w ratio was used, which has been previously employed for evaluating polymeric transfection agents.

In order to evaluate polymer-mediated transfection efficacy, luminescence (relative luminescence units or RLU) due to the expression of the luciferase protein was normalized to the protein content in each well, and the normalized values were compared to those determined for pEI-25. A number of polymers demonstrated statistically significant higher transfection efficacies than pEI-25 in the absence of serum. In particular, the 1,4 C-1,4 Bis polymer, based on the monomers, 1,4-cyclohexanedimethanol diglycidyl ether (1,4 C) and 1,4-bis(3-aminopropyl)piperazine, (1,4 Bis),demonstrated greatly (>80-fold) higher transfection efficacy than pEI-25 in the absence of serum. Other polymers demonstrated moderately higher (4-8 fold) or comparable transfection efficacies compared to that of pEI-25 in the absence of serum. For example, Neopentyl glycol diglycidyl ether-1,4-bis(3-aminopropyl)piperazine (NPGDE-1,4 Bis) and Ethylene glycol diglycidyl ether-1,4-bis(3-aminopropyl)piperazine (EGDE-1,4 Bis) polymers demonstrated approximately eight-fold higher efficacies than pEI-25. Polymers generated using 1,4-bis(3-aminopropyl)piperazine (1,4 Bis) as the amine monomer resulted in candidates with higher gene transfection efficacies than pEI-25.

The transfection efficacy of the 1,4 C-1,4Bis polymer was further evaluated as a function of polymer dose (i.e. polymer:pGL3 plasmid) in the absence of serum (FIG. 3b). While the polymer demonstrated comparable transfection efficacies to that of pEI-25 at low polymer: plasmid ratios (1:1 and 5:1), transfection efficacies with higher polymer ratios (10:1, 20:1 and 25:1) were significantly higher than those for pEI-25. In addition to the normalized ratios presented in FIG. 3b, the actual protein expression values (i.e. RLU/mg) were the highest observed in the experiments (not shown) when the 1,4C-1,4 Bis polymer was used, indicating that the polymer resulted in greater protein expression than pEI-25 under all conditions evaluated.

A sub-set of polymers that demonstrated appreciable transfection efficacies in the absence of serum was employed to transfect PC3-PSMA cells in the presence of serum-containing media. In the presence of serum, the transfection efficacy dropped in all polymers evaluated. Nevertheless, the 1,4 C-1,4 Bis polymer demonstrated considerably higher efficacies (approximately 30-fold) compared to pEI-25 in the presence of serum (FIG. 3c). The transfection efficacies of other polymers investigated were not statistically different compared to that of pEI-25.

Transfections were also carried out in serum-containing media with murine osteoblasts in order to compare the efficacy of the 1,4 C-1,4 Bis polymer to that of pEI-25 in cells unrelated to human prostate cancer cells. Lower transfection efficacies were observed in the MC3T3 murine osteoblast cell line for both polymers (not shown) as compared to PC3-PSMA cells, reflecting the challenges of transfecting these cells with non-viral transfection agents. However, as with the PC3-PSMA cells, the 1,4 C-1,4 Bis polymer was more effective (approximately 23-fold) than pEI-25 in transfecting these cells (FIG. 3d), indicating that the polymer might be useful in transfecting different cell types.

### Example 4

### Determining Cytotoxity of Polymer and Polyplexes

PC3-PSMA cells were seeded in a 24-well plate at a density of 50,000 cells/well and incubated overnight at 37° C. Different weight ratios of polymer-DNA polyplexes (10:1, 25:1, and 50:1 polymer: pGL3 plasmid) and different concentrations of polymers (4-20 ng/μL) were added in the absence of serum and the cells were incubated for 6 h to determine polyplex- and polymer-induced cytotoxicity, respectively. Following incubation, cells were treated with 100 μL of 4 μM ethidium homodimer-1 (EthD-1; Invitrogen) for 15 minutes and imaged immediately using Zeiss AxioObserver D1 inverted microscope (10×/0.3 numerical aperture (NA) objective; Carl Zeiss MicroImaging Inc., Germany). Fluorescence using excitation at 550 run and emission at 670 nm were used for the microscopy; dead/dying cells with compromised nuclei stained positive (red) for EthD-1.

Quantitative analysis of polymer/polyplex induced cell death was carried out as follows. The number of dead cells in each case was counted manually for three individual fields of fluorescence microscopy images by means of the Cell Counter plugin in ImageJ software (Rasband, W. S., ImageJ, U.S. National Institutes of Health, Bethesda, Md., USA, 1997-2005). The number of dead cells in both dead and live controls were determined for at least two fields of view and their average values were calculated. The number of red fluorescent cells in the case of each polymer or the corresponding polyplex was determined, and the percentage of dead cells was calculated by normalizing the number of dead cells in the sample to number of dead cells in the dead control.

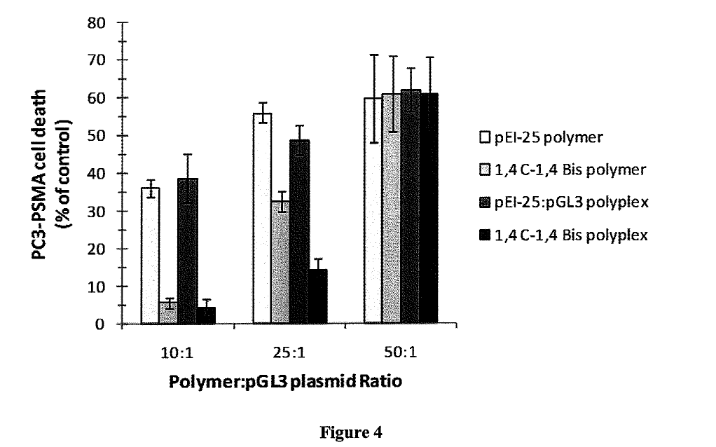

With reference to FIG. 4, pEI-25 was significantly more cytotoxic when compared to the 1,4 C-1,4 Bis polymer at concentrations of 4 ng/μL and 10 ng/μL (equivalent polymer: pGL3 plasmid ratios of 10:1 and 25:1). However, both polymers demonstrated similar cytotoxicities at 20 ng/μL (equivalent polymer: pGL3 plasmid ratios of 50:1). Interestingly, 1,4 C-1,4 Bis polymer based polyplexes (polymer:pGL3 plasmid ratios of 10:1 and 25:1) demonstrated lower cytotoxicities when compared to the polymer alone. In contrast, pEI-25 based polyplexes showed comparable cytotoxicity compared to the polymer alone at all polymer:pGL3 plasmid ratios investigated. The low cytotoxicities of the 1,4C-1,4 Bis polymer and its polyplex with pGL3 plasmid are believed to be in part responsible for the higher transfection efficacies of this polymer.

Phase contrast and fluorescence microscopy images were used to demonstrate the cytotoxicity of pEI-25 polymer, 1,4C-1,4Bis polymer, pEI-25: pGL3 polyplex, and 1,4C-1,4Bis:pGL3 polyplex toward PC3-PSMA cells in serum-free media

### Example 5

### Characterization of (1,4C-1,4 Bis)

Characterization experiments were carried out with polymer (1,4C-1,4Bis) that demonstrated successful cellular transfection. The polymer EGDE-3,3′ was also used in some cases in order to demonstrate the results for a different polymer used in transfections.

The disappearance of reactive (primary and secondary) amines with time was used to monitor the kinetics of the diglycidyl ether-polyamine reaction (FIG. 1a); the ninhydrin assay (Friedman, M. Applications of the ninhydrin reaction for analysis of amino acids, peptides, and proteins to agricultural and biomedical sciences. Journal of Agricultural and Food Chemistry 2004, 52 (3), 385-406.60) was used to determine the concentration of reactive amines at each time point. The ninhydrin assay results in a yellow-orange color in the case of secondary amines and a dark blue/purple color in the case of primary amines. Approximately 2 mg of the polymers were weighed into 1.5 mL microcentrifuge tubes (Fisher) at different time points (0-24 h) during the polymerization reaction. Ninhydrin reagent (Sigma; 100 μL) and DI water (200 μL) were added to the polymers in the centrifuge tubes, following which the tubes were placed in a boiling water bath for 10 min and cooled to room temperature (22° C.). The mixture was diluted by adding 500 μL of 95% ethanol. The mixtures were further diluted 10- and 100-fold using DI water in order to obtain absorbance values within the calibration range (using glycine standards) employed. Absorbance was measured at 570 nm in triplicate for each sample using a microplate reader (BioTek Synergy 2). The amine concentration was monitored every 4 h, and the concentration of amines in the reaction mixture at a given time point was normalized with the concentration of amines at the start of the reaction (t=0) in order to obtain percentage amine values.

The amine concentration remained largely invariant after 12 h of reaction time as determined from the using absorbance spectroscopy based on the formation of dark blue/purple color following reaction with primary amines. These results indicate that a reaction time of 16 h was appropriate for the generation of polymers that demonstrated moderate to high DNA binding and cellular transfection efficacies. In addition, the presence of residual primary amines (approximately 30% of the initial primary amine concentration, as reported by the dark blue color of the ninhydrin reaction), indicated the formation of branched polymers with multiple terminal primary amines. This was consistent with what can be expected from the employed reaction chemistry (FIG. 1a).

Gel permeation chromatography was employed to determine molecular weight values of the 1,4C-1,4Bis polymer which were as follows: Mn) 3.9 kDa and Mw) 23.5 kDa indicating a polydispersity (PD) of 5.96. These values indicated that the polymer molecular weights were comparable to those of pEI-25 used in the study: Mn) 10 kDa, Mw) 25 kDa, PD) 2.5 (Sigma). Polymer molecular weight was determined using a ViscoGEL column (MBLMW, Mixed Bed, dimensions: 7.8 mm x 30 cm) using 5% (v/v) acetic acid in water as the eluent (flow rate 1 mL/min) (Joshi, A.; Saraph, A.; Poon, V.; Mogridge, J.; Kane, R. S. Synthesis of potent inhibitors of anthrax toxin based on poly-Lglutamic acid. Bioconjugate Chem. 2006, 17 (5), 1265-1269.) The Mn and Mw values were estimated as an average of two experimental runs using a light scattering Viscotek 270 Trisec Dual Detector; OmniSEC software, λ) 670 nm.

Formation of the 1,4C-1,4Bis polymer was further verified using Fourier transform infrared (FT-IR) spectroscopy by following the appearance and disappearance of certain bands as a function of reaction time. FT-IR spectra were obtained at two different polymerization time points (t=0 and t=16 h) in order to ascertain the formation of the polymers. Polymer samples were loaded on a germanium attenuated total reflectance (GATR) crystal such that they covered the center area of the crystal. The sample chamber was equilibrated to approximately 4 mbar pressure in order to minimize interference from atmospheric moisture and CO₂. The absorption spectrum was measured between 650 and 4,000 cm-1 using a Broker IFS 66 v/S FT-IR spectrometer and the background spectrum was subtracted from all sample spectra.

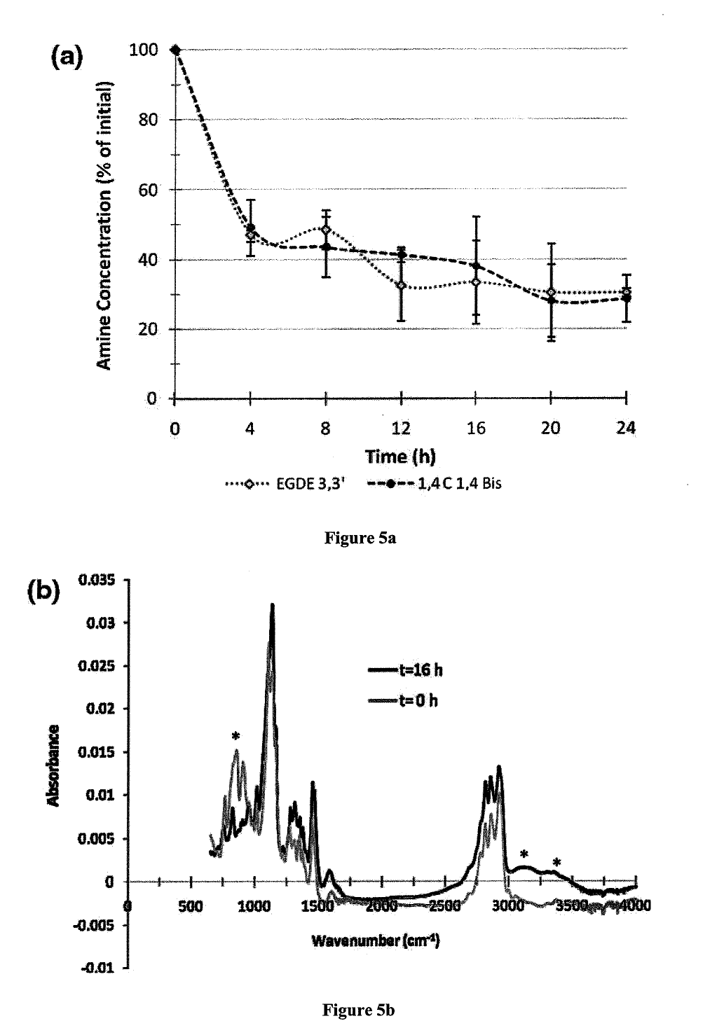

The epoxide peak ranging from 858 to 918 cm⁻¹ can be seen in the monomer mixture at time t=0 h (FIG. 5b) due to stretching and contraction of C—O bonds in the epoxide moiety. However, this peak is significantly reduced after 16 h of reaction time, indicating a reduction in the epoxide content upon formation of the cationic polymer (Loos, M. R.; Coelho, L. A. F.; Pezzin, S. H.; Amico, S. C. The effect of acetone addition on the properties of epoxy. Polimeros: Ciencia e Tecnologia 2008, 18 (1), 76-80). Characteristic spectral bands of primary amines were seen at 1100-1128 and 3358-3382 cm⁻¹ due to the presence of C—N and N—H bonds, respectively. The broad bands from 3382-3402 cm⁻¹ upon 16 h of reaction time can be attributed to hydroxyl (—OH) groups generated upon reaction of the epoxy rings with primary and secondary amines. Taken together, FT-IR analysis further confirmed that the diglycidyl ether-polyamine reaction resulted in the formation of the 1,4C-1,4Bis polymer that demonstrated high transfection efficacies in vitro.

What is claimed is:

1. A composition, comprising, a polymer of a diepoxide and an amine, wherein the diepoxide is
a. 1,4 butanediol diglycidyl ether (1,4 B);
b. 1,4-cyclohexanedimethanol diglycidyl ether (1,4 C);
c. 4-vinylcyclohexene diepoxide (4VCD);
d. ethyleneglycol diglycidyl ether (EDGE);
e. glycerol diglycidyl ether (GDE);
f. neopentylglycol diglycidyl ether (NPDGE);
g. poly(ethyleneglycol) diglycidyl ether (PEGDE); or
h. poly(propyleneglycol) diglycidyl ether (PPGDE); and
wherein the amine is
i. 1-(2-aminoethyl)piperidine;
j. 1,4-bis(3-aminopropyl)piperazine (1,4 Bis);
k. 3,3′-diamino-N-methyl dipropylamine (3,3′);
l. 4,7,10-trioxa-1,13-tridecanediamine;
m. aniline;
n. butylamine;
o. diethylenetriamine (DT);
p. ethylenediamine (ED);
q. N-(2-aminoethyl)-1,3-propanediamine (N-2amino); or
r. pentaethylenehexamine.

2. The composition of claim 1, wherein the polymer has a number average molecular weight (Mn) of from about 1 to about 20 kDa.

3. The composition of claim 1, wherein the polymer has a weight average molecular weight (Mw) of from about 10 to about 50 kDa.

4. The composition of claim 1, wherein the polymer has a polydispersity index (PDI) of from about 2 to about 7.

5. The composition of claim 1, wherein the polymer is cationic.

6. The composition of claim 1, further comprising a substance to be delivered into a cell.

7. The composition of claim 6, wherein the substance is a nucleic acid.

8. The composition of claim 1, wherein the polymer is poly(ethyleneglycol diglycidyl ether)-co-(3,3′-diamino-N-methyl dipropylamine) or poly(1,4-cyclohexanedimethanol diglycidyl ether)-co-[1,4-bis(3-aminopropyl)piperazine].

9. A pharmaceutical composition, comprising, a polymer of a diepoxide and an amine, wherein the diepoxide is
a. 1,4 butanediol diglycidyl ether (1,4 B);
b. 1,4-cyclohexanedimethanol diglycidyl ether (1,4 C);
c. 4-vinylcyclohexene diepoxide (4VCD);
d. ethyleneglycol diglycidyl ether (EDGE);
e. glycerol diglycidyl ether (GDE);
f. neopentylglycol diglycidyl ether (NPDGE);
g. poly(ethyleneglycol) diglycidyl ether (PEGDE); or
h. poly(propyleneglycol) diglycidyl ether (PPGDE); and
wherein the amine is
i. 1-(2-aminoethyl)piperidine;
j. 1,4-bis(3-aminopropyl)piperazine (1,4 Bis);
k. 3,3′-diamino-N-methyl dipropylamine (3,3′);
l. 4,7,10-trioxa-1,13-tridecanediamine;
m. aniline;
n. butylamine;
o. diethylenetriamine (DT);
p. ethylenediamine (ED);
N-(2-aminoethyl)-1,3-propanediamine (N-2amino); or
r. pentaethylenehexamine; and
and a substance to be delivered into a cell.

10. The pharmaceutical composition of claim 9, wherein the polymer has a number average molecular weight (Mn) of from about 1 to about 20 kDa.

11. The pharmaceutical composition of claim 9, wherein the polymer has a weight average molecular weight (Mw) of from about 10 to about 50 kDa.

12. The pharmaceutical composition of claim 9, wherein the polymer has a polydispersity index (PDI) of from about 2 to about 7.

13. The pharmaceutical composition of claim 9, wherein the polymer is cationic.

14. The pharmaceutical composition of claim 9, wherein the substance is a nucleic acid.

15. A method for delivering a nucleic acid into a cell, comprising, contacting a cell with i.) a polymer of a diepoxide and an amine; and ii.) a nucleic acid;
wherein the diepoxide is
a. 1,4 butanediol diglycidyl ether (1,4 B);
b. 1,4-cyclohexanedimethanol diglycidyl ether (1,4 C);
c. 4-vinylcyclohexene diepoxide (4VCD);
d. ethyleneglycol diglycidyl ether (EDGE);
e. glycerol diglycidyl ether (GDE);
f. neopentylglycol diglycidyl ether (NPDGE);
g. poly(ethyleneglycol) diglycidyl ether (PEGDE); or
h. poly(propyleneglycol) diglycidyl ether (PPGDE); and
wherein the amine is
i. 1-(2-aminoethyl)piperidine;
j. 1,4-bis(3-aminopropyl)piperazine (1,4 Bis);
k. 3,3′-diamino-N-methyl dipropylamine (3,3′);
l. 4,7,10-trioxa-1,13-tridecanediamine;
m. aniline;
n. butylamine;
o. diethylenetriamine (DT);
p. ethylenediamine (ED);
q. N-(2-aminoethyl)-1,3-propanediamine (N-2amino); or
r. pentaethylenehexamine.

16. The method of claim 15, wherein the polymer has a weight average molecular weight (Mw) of from about 10 to about 50 kDa.

17. The method of claim 15, wherein the polymer has a polydispersity index (PDI) of from about 2 to about 7.

18. The method of claim 15, wherein the polymer is cationic.

19. The method of claim 15, wherein the polymer is poly(ethyleneglycol diglycidyl ether)-co-(3,3′-diamino-N-methyl dipropylamine) or poly(1,4-cyclohexanedimethanol diglycidyl ether)-co-[1,4-bis(3-aminopropyl)piperazine].

20. The method of claim 15, wherein the polymer and the nucleic acid together form a polyplex.
